(19) United States
(12) Patent Application Publication
(10) Pub. No.: US 2025/0197739 A1
(43) Pub. Date: Jun. 19, 2025

(54) CONVERSION OF CARBON DIOXIDE TO GASOLINE USING E-SMR

(71) Applicant: TOPSOE A/S, Kgs. Lyngby (DK)

(72) Inventors: Sudip DE SARKAR, Kgs. Lyngby (DK); Kim AASBERG-PETERSEN, Kgs. Lyngby (DK)

(73) Assignee: TOPSOE A/S, Kgs. Lyngby (DK)

(21) Appl. No.: 18/852,193

(22) PCT Filed: Mar. 31, 2023
(86) PCT No.: PCT/EP2023/058438

§ 371 Date: Sep. 27, 2024

(30) Foreign Application Priority Data
Apr. 1, 2022 (EP) 22166260.4

(51) Int. Cl. C10G 55/02

(52) U.S. Cl. CPC C10G 55/02; C10G 2300/104; C10G 2300/202; C10G 2400/02

(57) ABSTRACT

A system and process are provided for reforming, in particular, a propane and/or butane rich stream, e.g. a liquified petroleum gas, LPG feed. A first stream being rich in propane and/or butane is hydrogenated, desulfurised, pre-reformed and then subjected to electrical steam reforming. A gasoline synthesis plant, incorporating said system is also provided. The invention provides an overall more efficient feed-to-gasoline system and process.

### TECHNICAL FIELD

The present invention relates to a more efficient sustainable feed-to-gasoline system and process, where electrical SMR is used to improve gasoline yield by recycling propane and/or butane rich streams and/or off-gas streams.

### BACKGROUND

Processes for the conversion of sustainable feeds, such as CO₂, biomass etc. to gasoline via methanol are known. Biomass can first be converted to syngas via gasification followed by conversion of said syngas to methanol and finally methanol conversion to gasoline. CO₂ feed, together with H₂ feed, can be converted to methanol followed by conversion of said methanol to gasoline. Irrespective of the main feed, there are some by-products along with gasoline. One of the by-products from such processes is a fraction containing lighter hydrocarbons such as propane and/or butane—this fraction is known as liquified petroleum gas, LPG. Off-gas streams comprising CO₂, H₂, CH₄, higher hydrocarbons etc. are also typically produced.

LPG is itself considered to have little or no commercial value. Moreover, the off-gas streams often have no efficient use, apart from using them in fired equipment, which causes CO₂ emission. It would, therefore, be of interest to recycle these product streams as part of the gasoline synthesis process itself, in order to improve overall C-efficiency of this process. Furthermore, recycling propane and/or butane rich stream and/or off-gas stream via reforming in CO₂ and H₂ feed based methanol plant enhances methanol loop performance.

An LPG stream and/or off-gas streams can be subjected to a traditional reforming process, such as steam methane reforming, and the reformed synthesis gas stream can be recycled to the methanol loop. However, if a hydrocarbon fuel were used in an LPG reforming process, it could result in CO₂ emissions. If a hydrogen fuel were used in an LPG reforming process, consumption of valuable and expensive H₂ could result.

In gasoline synthesis from methane, the plant should already comprise a reformer and, thus, LPG and/or off-gas streams could be directed there.

However, plants/systems for gasoline synthesis from sustainable feeds and/or feeds from biogas gasification and/or mixtures comprising CO₂ and H₂ do not comprise a reformer. U.S. Pat. No. 4,520,216A discloses a process for synthetic hydrocarbons, especially high-octane gasoline, from synthesis gas by catalytic conversion in two steps.

WO2007108014 A1 discloses a process and system for producing gasoline or diesel from carbon dioxide and water. A reforming unit downstream of and in fluid communication with the gasoline or diesel generation unit is arranged to e.g. steam-reform a recycle stream having a significant portion of LPG and fuel gas, namely 15-40 wt % of the liquid product.

US20160168476 discloses a methanol-to-gasoline plant in which a by-product stream is withdrawn and converted to a synthesis gas in a reformer. This synthesis gas is combined with a main synthesis gas and fed to a first reactor for conversion to methanol. LPG and similar off-gases are directed away from the reformer.

US2021395083 discloses a system for hydrogen production in an electrical membrane reformer from a hydrocarbon feed which may include LPG. The electrical membrane reformer produces two separate streams, i.e. a hydrogen stream and a carbon dioxide stream.

A need therefore exists for an effective process and system for utilising the LPG and/or off-gas by-product streams from a sustainable feed-to-gasoline synthesis plant to improve overall C-efficiency, but in which disadvantages can be avoided; in particular, in which additional CO₂ emissions are avoided.

### SUMMARY

It has been determined that LPG and/or off-gas stream recycle can be enabled to provide higher efficiency of sustainable feed to gasoline conversion. With the proposed plant layout, this can be achieved with no or significantly lower CO₂ emission compared to traditional processes for a similar purpose. Furthermore, the proposed layout also has provided a possibility of reducing the consumption of hydrogen feedstock, the production of which is power consuming and capital cost intensive, e.g. when using an electrolysis unit for producing the hydrogen. Thus, the reduction of power consumption in the electrolysis unit more than outweighs the power consumption in the e-SMR resulting in a reduction of power consumption for the overall system.

A system is therefore provided for reforming a first stream, being a propane and/or butane rich stream, said system comprising:
a first stream, being rich in propane and/or butane;
an electrical steam methane reformer (e-SMR), arranged to receive the first stream and carry out an electrical steam methane reforming (e-SMR) step, so as to provide a first syngas stream.

Throughout the application, the term “being rich in propane and/or butane” means comprising at least 50% propane and/or butane.

Throughout the application, the term “so as to provide a first syngas stream” means that the e-SMR provides one product stream. The outlet of the e-SMR is a one product stream, which is implicit in an e-SMR.

Accordingly, the system for reforming a first propane and/or butane rich stream, comprises:
a first stream, being rich in propane and/or butane by said first stream comprising at least 50% propane and/or butane;
an electrical steam methane reformer (e-SMR), arranged to receive the first stream and carry out an electrical steam methane reforming (e-SMR) step, so as to provide one product stream in the form of a first syngas stream.

A process for reforming a first stream, being rich in propane and/or butane, is provided, using the above-mentioned system.

Also provided is a gasoline synthesis plant, which comprises the above-mentioned system, as well as a process for gasoline synthesis from sustainable feeds, in such a plant.

Further details of the technology are provided in the enclosed dependent claims, figures and examples.

### LEGENDS TO THE FIGURES

The technology is illustrated by means of the following schematic illustrations, in which:

FIG. 1 shows a simple layout of one embodiment of the system of the invention.

FIG. 2 shows a more developed layout of the system of the invention.

FIG. 3 shows a gasoline plant, comprising the system of the invention.

FIG. 4 shows another gasoline plant, comprising the system of the invention.

FIG. 5 shows yet another gasoline plant, comprising the system of the invention.

### DETAILED DISCLOSURE

Unless otherwise specified, any given percentages for gas content are % by volume. All feeds are preheated as required.

The term “synthesis gas” (abbreviated to “syngas”) is meant to denote a gas comprising hydrogen, carbon monoxide, carbon dioxide and small amounts of other gasses, such as argon, nitrogen, methane, etc.

A “sustainable feed” may be a CO₂ feed, a H₂ feed or a biomass feed.

The term “reforming” and “steam reforming” may be used interchangeably.

The term “at least a portion” of a given stream, means the entire stream or a portion thereof.

The terms “system”, “plant” i.e. process plant, are used interchangeably. Throughout this specification, the term “system for reforming” is used interchangeably with the term “reforming system” or it is simply referred to as “system 100” in which the ref. number 100 is per the appended figures.

The terms “section” and “unit” refers normally in this specification to a subset of a plant or system.

The term “suitably” may be given the same meaning as “optionally”, i.e. an optional embodiment.

The term “invention” may be used interchangeably with the term “application”.

The use of the article “an” in connection with an item such as a unit means “one or more”. For instance, the term “an electrical steam methane reformer (e-SMR)” means one or more, such as a plurality of e-SMRs arranged in parallel.

Other definitions are provided throughout the patent application in connection with the recital of embodiments.

In a first embodiment, a system is provided for electrical steam reforming a first stream rich in propane and/or butane (e.g. a liquified petroleum gas, LPG stream), which improves gasoline product yield, while avoiding excess CO₂ emissions.

The system comprises a first stream being rich in propane and/or butane. The term “rich in propane and/or butane” means that at least 50%, such as at least 60%, preferably at least 75% of this first stream is propane and/or butane. Typically, LPG contains 70-80 vol % butane, 20-30 vol % propane and some other hydrocarbons.

The first stream is—in one preferred aspect—an LPG feed. LPG is typically a mix of lighter hydrocarbons, such as propane and butane. Propylene, butylenes and various other hydrocarbons are usually also present in LPG in small concentrations such as C₂H₆, CH₄ etc. An LPG feed may also comprise olefins. In one aspect of the invention, the first stream is an LPG feed derived from a gasoline synthesis plant or refinery.

Accordingly, in an embodiment, the first stream is an LPG stream.

In an embodiment, the system further comprises a second stream being an off-gas stream comprising CO₂, H₂ and CH₄, said second stream being arranged to be mixed with the first stream, upstream the inlet of the e-SMR.

In an embodiment, the system further comprises a separation section, arranged to receive at least a portion of said first syngas stream and separate it into at least a second syngas stream and a process condensate.

By the invention, the outlet of the system (reforming system) is a one product stream which comprises CO. A syngas stream comprising CO is advantageous for the methanol synthesis. The addition of syngas via reforming of said first reforming feed stream increases the CO content in the inlet to the methanol synthesis unit. This is advantageous for improving the performance of the methanol synthesis unit, for instance where this unit is provided as a methanol (MeOH) synthesis loop, i.e. smaller MeOH loop compared to when main feed to the methanol synthesis unit is H₂ and CO₂. A higher molar ratio CO/CO₂ to the methanol synthesis unit enables a lower catalyst volume, less water formation and thus less need for purification downstream to remove it, and thereby also a smaller methanol synthesis unit e.g. MeOH loop. More specifically, the provision of much higher content of CO with respect of CO₂ in the syngas feed, with the molar ratio CO/CO₂ being greater than 1, such as greater than 2, for instance a ratio of 10 or higher, means a more reactive syngas, since it enables that the methanol reaction proceeds with low generation of water which is detrimental for the methanol synthesis catalyst of the methanol synthesis unit, as the methanol synthesis is conducted mainly according to the reaction: CO+2H₂=CH₃OH, rather than typically via the reaction 3H₂+CO₂═CH₃OH+H₂O. Less hydrogen consumption, by a drastic decrease now requiring 2 moles of H₂ instead of 3 moles of H₂ for each mole of produced methanol, is also achieved. The resulting water has also a negative effect on the performance of the methanol synthesis catalyst and the catalyst volume may increase with more than 100% if the CO₂ concentration in the syngas feed is too high, e.g. 90%. Much more energy is also required for the downstream purification of the raw methanol because all the water is normally removed by distillation. Not least, there is an increase in the output of methanol being produced, which manifests itself in a higher yield of the desired gasoline product.

In one aspect, the system may comprise a hydrogenation section arranged to receive the first stream, and provide a hydrogenated first stream. In the hydrogenation section, the first stream is mixed with a hydrogen feed, and passed over a catalyst active in hydrogenation. The hydrogenation section may comprise one or more hydrogenation reactors in series. Hydrogenation converts unsaturated hydrocarbon components such as propylene or butylene to the corresponding saturated hydrocarbons, which can reduce or avoid carbon formation (in a reforming step) by converting olefins into alkanes. Hydrogenation catalysts and reactors suitable for such processes are commercially available and known to the skilled person.

Accordingly, in an embodiment, the system of the invention further comprises:
a hydrogenation section hydrogenating the first stream to provide a hydrogenated first stream;
optionally a desulfurisation section for desulfurising said hydrogenated first stream to provide a desulfurised first stream;
optionally a pre-reforming section for pre-reforming the first stream to provide a pre-reformed first stream;

wherein the electrical steam methane reformer (e-SMR) is arranged to receive: the first stream, or the hydrogenated first stream, or optionally the desulfurised first stream, or optionally the pre-reformed first stream; and carry out said electrical steam methane reforming (e-SMR) step, so as to provide said first syngas stream (41), i.e. so as to provide said one product stream in the form of a first syngas stream.

The system may also comprise a desulfurisation section arranged to receive the hydrogenated first stream and provide a desulfurised first and/or second stream. Typically, the desulfurisation section comprises one or more hydrodesulfurization (HDS) reactors. Desulfurisation converts sulfur-containing compounds in the first stream to hydrocarbons (typically saturated hydrocarbons) and sulfur-containing compounds (e.g., H₂S) as by-product. This can reduce catalyst poisoning in subsequent conversion steps. Desulfurisation catalysts and reactors suitable for such processes are commercially available and known to the skilled person. Substances other than sulfur that might need to be removed in such a purification step include chlorine, dust and heavy metals.

A pre-reforming section may be arranged to receive the first stream and carry out a pre-reforming step. Hence, an electrical steam methane reformer (e-SMR) may be arranged alone or together with an upstream pre-reformer. A pre-reformed stream is provided. Pre-reforming is an additional reforming step, which allows a syngas with a desired composition to ultimately be obtained, i.e. in which higher hydrocarbons are converted to methane. Pre-reforming suitably takes place at ca. 350-700° C. to convert higher hydrocarbons as an initial step. Pre-reforming catalysts and reactors suitable for such processes are commercially available and known to the skilled person. Pre-reformer units (prereformers) used in the present invention are catalyst-containing reactor vessels, and are typically adiabatic. In the pre-reforming units, heavier hydrocarbon components in the hydrocarbon feedstock are steam reformed and the products of the heavier hydrocarbon reforming are methanated. The skilled person can construct and operate suitable pre-reformer units as required. Pre-reformer units suitable for use in the present system/process are provided in applicant's co-pending applications EP20201822 and EP21153815. The pre-reformed stream comprises methane, hydrogen, carbon monoxide and also carbon dioxide. The pre-reformed stream at the outlet of the prereformer may be in the temperature range: 400° C.-500° C.

The system comprises an electrical steam methane reformer (e-SMR). The e-SMR requires a feed of steam. The e-SMR receives the first stream and carries out an electrical steam methane reforming (e-SMR) step, and thereby provides a first syngas stream. E-SMRs use electrical resistance heating to provide sufficient heating of the reactant stream and catalyst for effective reforming reaction to be carried out. The e-SMR preferably comprises a pressure shell housing a structured catalyst, wherein the structured catalyst comprises a macroscopic structure of an electrically conductive material. The macroscopic structure supports a ceramic coating, where said ceramic coating supports a catalytically active material. The reforming step comprises the step of supplying electrical power via electrical conductors connecting an electrical power supply placed outside said pressure shell to said structured catalyst, allowing an electrical current to run through said macroscopic structure material, thereby heating at least part of the structured catalyst to a temperature of at least 500° C. The e-SMR of the invention is a non-membrane type e-SMR, i.e. it is not a membrane reformer producing two or more effluent streams, e.g. a reformer with a hydrogen selective membrane producing a hydrogen-rich product stream and a carbon dioxide rich product stream. The e-SMR of the invention produces one product stream in the form of a first syngas stream with one composition. Suitably, the electrical power supplied to the electrically heated reformer is generated by means of a renewable energy source. Suitable electrical steam reformers for use in the electrical steam reformer section of the present invention are as disclosed in co-pending applications WO2019228797 and WO/2019/228798. In a steam reforming process, a stream of hydro-carbons and steam is catalytically reformed to a product stream of hydrogen and carbon oxides; typified by the following reactions:

$$\begin{matrix}{CH}_{4} + {H}_{2} O \to CO + 3 {H}_{2} & Δ H {°}_{298} = - 49.3 kcal / mole \\ {CH}_{4} + 2 {H}_{2} O \to {CO}_{2} + 4 {H}_{2} & Δ H {°}_{298} = - 39.4 kcal / mole\end{matrix}$$

Suitable process conditions (temperatures, pressures, flow rates etc.) and suitable catalysts for such steam reforming processes are known in the art.

The composition of this first syngas stream from the e-SMR is typically (by volume):
40-70% H₂ (dry)
10-30% CO (dry)
2-20% CO₂ (dry)
0.5-5% CH₄ (dry)

Use of an e-SMR in this manner allows recycling of the LPG streams and/or off-gas streams, such that additional CO₂ emissions can be avoided, or significantly minimised.

Suitably, a separation section is arranged in the system, to receive the first syngas stream and separate it into at least a second syngas stream and a process condensate. This separation section advantageously removes water from the first syngas stream.

As the first syngas stream is at an elevated temperature (e.g. 900-1100° C.) at the outlet of the e-SMR, it can advantageously be heat-exchanged with upstream components in the system, for effective energy use in the system. The system may therefore comprise one or more heat exchangers, being arranged to provide heat exchange between the first syngas stream and one or more of: the first stream, the desulfurised first stream and boiler feed water stream.

Suitably, the first syngas stream is heat exchanged with the desulfurised first stream first, then a boiler feed water stream, and then with the first stream. Alternatively, or additionally, one or more electrical heaters may be used to raise the temperature of one or more of: the first stream, the hydrogenated first stream, the desulfurised first stream and boiler feed water stream.

As recited above, the system may further comprise a second stream being an off-gas stream comprising CO₂, H₂ and CH₄, said second stream being arranged to be mixed with the first stream, upstream the inlet of the e-SMR. The second stream being an off-gas stream comprising CO₂, H₂ and CH₄ may be any off-gas stream, i.e. from any off-gas producing unit, comprising CO₂, H₂ and CH₄, optionally an off-gas stream comprising CO₂, H₂ and CH₄ generated within or outside a plant comprising the system of the invention. In a particular embodiment, the second stream being an off-gas stream comprising CO₂, H₂ and CH₄ is an off-gas stream generated within a plant comprising the system of the invention. In a particular embodiment, the second stream being an off-gas stream comprising CO₂, H₂ and CH₄ is an off-gas stream generated within a gasoline synthesis plant comprising the system of the invention.

In an embodiment, the system i.e. the reforming system may further comprise a hydrogen recovery section. The hydrogen recovery section is arranged to receive at least a portion of the second syngas stream and provide at least a hydrogen-rich stream and a third syngas stream. The hydrogen recovery section may comprise a membrane hydrogen separation unit or a PSA (pressure swing adsorption) unit or both. Suitably, at least a portion of the second syngas stream and at least a portion of the third syngas stream are arranged to be combined to a combined syngas stream.

At least a portion of the hydrogen-rich stream obtained from the hydrogen recovery section and/or a portion of the second syngas stream from the separation section may be used in the hydrogenation section. Therefore, in an embodiment, at least a portion of the hydrogen-rich stream may be combined with the first feed and/or off-gas feed, upstream the hydrogenation section. Alternatively, or additionally, recovered H₂ can also be used in a hydrocracking section downstream a gasoline synthesis, or used as hydrogen source in the upgrading section of the gasoline synthesis plant, for instance in a hydroisomerisation (HDI) reactor and/or hydrocracking (HCR) reactor therein.

The invention provides also a process for reforming a first stream being rich in propane and/or butane, said process comprising the steps of:
providing a system according to any one of above embodiments;
optionally, hydrogenating the first stream in the hydrogenation section (10) to provide a hydrogenated first stream;
optionally, desulfurising said hydrogenated first stream in desulfurisation section (20), to provide a desulfurised first stream;
optionally, pre-reforming the first stream in pre-reforming section (30), to provide a pre-reformed first stream;
performing an electrical steam methane reforming (e-SMR) step on said first stream in an electrical steam methane reformer (e-SMR, 40), to provide a first syngas stream.

The system set out above may be used with any suitable LPG source and/or off-gas source, e.g. a gasoline refinery. In particular, however, the system is useful in a sustainable feed-to-gasoline plant. A gasoline synthesis plant is therefore provided, which comprises the system described herein.

The gasoline synthesis plant according to this aspect comprises:
a CO₂ rich feed comprising CO₂ to said plant,
a H₂ rich feed comprising H₂ to said plant,
a methanol synthesis unit, arranged to receive the CO₂ rich feed and the H₂ rich feed, and provide an effluent stream comprising methanol;
a gasoline synthesis section, arranged to receive at least a portion of the effluent stream comprising methanol, and provide a raw product containing hydrocarbons boiling in the gasoline range;
an upgrading section, arranged to receive at least a portion of the raw product from the gasoline synthesis section, and provide a gasoline product stream; and a first stream being rich in propane and/or butane, and/or a second stream being an off-gas stream; optionally, said upgrading section comprising: a de-ethanizer for providing at least a portion of said second stream, LPG splitter for providing said first stream, optionally a hydroisomerisation (HDI) reactor and/or a hydrocracking (HCR) reactor;
said gasoline synthesis plant further comprising the system as described herein,
wherein said system is arranged to receive at least a portion of said first stream from the upgrading section as first stream to said system,
and/or wherein said system is arranged to receive at least a portion of said second stream from the upgrading section,
and provide a first syngas stream from said first and/or said second stream,
wherein the plant further comprises in said system a separation section, arranged to receive at least a portion of said first syngas stream and separate it into at least a second syngas stream and a process condensate, and wherein at least a portion of said second syngas stream is arranged to be fed to the inlet of the methanol synthesis unit, preferably in admixture with said CO₂ rich feed and/or said H₂ rich feed.

The plant therefore comprises, in general terms:
a CO₂ rich feed to said plant,
a H₂ rich feed to said plant,
a methanol synthesis unit;
a gasoline synthesis section;
an upgrading section; and
the system, as set out above.

The upgrading section comprises a de-ethanizer for providing at least a portion of said second stream, LPG splitter for providing said first stream, optionally a hydroisomerisation (HDI) reactor and/or a hydrocracking (HCR) reactor. As used herein, the upgrading section comprises a distillation section which includes said de-ethanizer and said LPG splitter. As is well-known in the art, a conventional technology for gasoline synthesis from oxygenates such as methanol involves plants comprising a MTG section (methanol-to-gasoline section) and a downstream distillation section. The MTG section may be provided as a MTG loop and comprises: a MTG reactor; a product separator for withdrawing a bottom water stream, an overhead recycle stream from which an optional fuel gas stream may be derived, as well as a raw gasoline stream comprising C2 compounds, C3-C4 paraffins (LPG) and C5+ hydrocarbons (gasoline boiling components); and a recycle compressor for recycling the overhead recycle stream by combining it with the oxygenate feed stream, e.g. methanol feed stream. The overhead recycle stream (or simply, recycle stream) acts as diluent, thereby reducing the exothermicity of the oxygenate conversion. In the distillation section, C2 compounds are removed in the de-ethanizer, such as de-ethanizer column, and then a C3-C4 fraction is removed as LPG as the overhead stream in a LPG splitter, such as a LPG-splitting column, while stabilized gasoline is withdrawn as the bottoms product. The stabilized gasoline or the heavier components of the stabilized gasoline, such as the C9-C11 fraction may optionally be further treated and thereby refined, e.g. by conducting hydroisomerization (HDI) into an upgraded gasoline product, i.e. as a gasoline product stream. Optionally, hydrocracking (HCR) may also be conducted. HDI and HCR reactors and conditions are well-known in the art.

The CO₂ rich feed is provided to the methanol synthesis unit (also, in an embodiment, called a methanol loop). The CO₂ rich feed suitably comprises more than 90% CO₂, preferably more than 95% CO₂, preferably more than 99% CO₂. The CO₂ rich feed may in addition to CO₂ comprise minor amounts of, for example, steam, oxygen, nitrogen, oxygenates, amines, ammonia, carbon monoxide, and/or hydrocarbons. The CO₂ rich feed suitably comprises only low amounts of hydrocarbon, such as for example less than 5% hydrocarbons or less than 3% hydrocarbons or less than 1% hydrocarbons.

The H₂ rich feed is provided to the methanol synthesis unit. Suitably, the H₂ rich feed consists essentially of hydrogen. The H₂ rich feed of hydrogen is suitably “hydrogen rich” meaning that the major portion of this feed is hydrogen; i.e. over 75%, such as over 85%, preferably over 90%, more preferably over 95%, even more preferably over 99% of this feed is hydrogen. One source of the H₂ rich feed of hydrogen can be one or more electrolyser units. In addition to hydrogen this feed may for example comprise steam, nitrogen, argon, carbon monoxide, carbon dioxide, and/or hydrocarbons. In some cases, a minor content of oxygen may be present in this H₂ rich feed, typically less than 100 ppm. The H₂ rich feed suitably comprises only low amounts of hydrocarbon, such as for example less than 5% hydrocarbons or less than 3% hydrocarbons or less than 1% hydrocarbons.

The CO₂ rich feed and the H₂ rich feed are-in one aspect-combined prior to being fed to the methanol synthesis unit.

In this embodiment, the gasoline synthesis plant comprises a methanol synthesis unit, being arranged to receive CO₂ rich and H₂ rich feeds as well as the second syngas. An effluent stream comprising methanol is obtained. The process of converting the CO₂ rich and H₂ rich streams can occur, for example by compressing them and sending the compressed, combined gas through a boiling water reactor, where at least a portion of the CO, CO₂ and H₂ is converted to methanol followed by a condensation section separating the purge gas stream from the methanol in a liquid phase.

In another embodiment, a gasoline synthesis plant is provided, comprising:
a syngas feed from biomass gasification to said plant,
optionally, a H₂ rich feed comprising H₂ to said plant,
a methanol synthesis unit, arranged to receive the syngas feed and optionally, the H₂ rich feed, and provide an effluent stream comprising methanol;
a gasoline synthesis section, arranged to receive at least a portion of the effluent stream comprising methanol, and provide a raw product containing hydrocarbons boiling in the gasoline range;
an upgrading section, arranged to receive at least a portion of the raw product from the gasoline synthesis section, and provide a gasoline product stream; and a first stream being rich in propane and/or butane, and/or a second stream being an off-gas stream; optionally, said upgrading section comprising: a de-ethanizer for providing at least a portion of said second stream (2, 253), LPG splitter for providing said first stream (1, 242), optionally a hydroisomerisation (HDI) reactor, optionally also a hydrocracking (HCR) reactor;
said gasoline synthesis plant further comprising the system as described herein,
wherein said system is arranged to receive at least a portion of said first stream from the upgrading section as first stream to said system,
and/or wherein said system is arranged to receive at least a portion of said second stream from the upgrading section as second stream to said system,
and provide a first syngas stream from said first and/or said second stream,
wherein the plant further comprises a separation section, arranged to receive at least a portion of said first syngas stream and separate it into at least a second syngas stream and a process condensate, and wherein at least a portion of said second syngas stream is arranged to be fed to the inlet of the methanol synthesis unit, preferably in admixture with said syngas feed and/or said H₂ rich feed.

This plant comprises, in general terms:
a syngas feed from biomass gasification to said plant,
optionally, a H₂ rich feed to said plant
a methanol synthesis unit;
a gasoline synthesis section;
an upgrading section; and
the system, as set out above.

In this embodiment, the gasoline synthesis plant comprises a methanol synthesis unit, being arranged to receive syngas feed from biomass gasification and optionally, H₂ rich feed. An effluent stream comprising methanol is obtained. The process of converting the syngas feed from biomass gasification and optionally, H₂ rich streams can occur, for example by compressing them and sending the compressed, combined gas through a boiling water reactor, where at least a portion of the CO, CO₂ and H₂ is converted to methanol followed by a condensation section separating the purge gas stream from the methanol in a liquid phase.

The raw methanol stream (i.e., the effluent stream comprising methanol) comprises a major portion of methanol; i.e. over 50 wt %, such as over 75 wt %, preferably over 85 wt %, more preferably over 90 wt % of this feed is methanol. Other minor components of this stream include but not limited to, higher alcohols, ketones, aldehydes, DME, organic acids and dissolved gases.

To obtain an optimized yield in the methanol production, the stoichiometry of H₂, CO and CO₂ needs to be considered. In a preferred embodiment, the stoichiometry of H₂, CO and CO₂ in the first and second syngas streams falls within an interval such that the first and second syngas streams have a module between 1.8 and 2.2, preferably between 1.95 and 2.1, where the module is defined in terms of molar content:

$$M = ( {H}_{2} - {CO}_{2} ) / ( CO + {CO}_{2} ) .$$

In one embodiment, a water separation unit is located between the methanol synthesis unit and the gasoline synthesis section, for instance upstream a methanol storage tank as recited in a below embodiment. This is advantageous when methanol is produced from CO₂ and H₂, as the effluent comprising methanol obtained from such feeds contains relatively high volumes of water (e.g. up to 50% water).

In an embodiment, a methanol storage tank is arranged between said methanol synthesis unit and said gasoline synthesis section, i.e. downstream the methanol synthesis unit and upstream the gasoline synthesis section, for storing at least a portion of the effluent stream comprising methanol.

This provides a simple solution for coping with intermittent sources for producing the electricity required in upstream electrolysis and/or the e-SMR. The methanol storage tank may be arranged downstream said water separation section for removing water. The water separation section is for instance a distillation column. The methanol storage tank accumulates the methanol at low pressure, such as less than 5 barg, for instance atmospheric pressure, thus enabling the use of inexpensive materials for such storage tank while also serving as efficient buffer for any sudden variations in electricity due to the intermittent nature of the source producing it, such as wind and solar energy.

The plant (and process) according to the present invention thus enables not only improving hydrogen (H) and carbon (C) efficiency of the plant, while improving performance and thereby reducing the size of the methanol synthesis unit, for instance a MeOH-loop, but at the same time provides a robust plant which copes with the sudden and often huge variations in electricity supply, and which electricity is required for e.g. upstream electrolysis of water or steam into the hydrogen required in the syngas feed for methanol production.

In an embodiment, the methanol synthesis unit is arranged for the first syngas stream being up to up to 50% by volume basis, such as 5-45%, for instance 15-45%, e.g. 10-40% or 20-40% of the inlet of the methanol synthesis unit. The first syngas stream may thus be 5, 10, 15, 20, 25, 30, 35, 40, 45, 50% volume basis of the inlet of the methanol synthesis unit.

The particular feeding point of the syngas from the reforming system to the inlet of the methanol synthesis unit is, for instance, downstream the mixing point of said CO₂ rich feed and/or said H₂ rich feed and upstream the first syngas feed compressor arranged therein, thus in admixture with said first syngas feed, or in admixture with said second syngas feed. The H₂ rich feed from e.g. water electrolysis is provided by a dedicated H₂-compressor. The CO₂ rich feed, suitably after CO₂-gas cleaning, is combined with the H₂ rich feed into said first syngas feed and provided to the methanol reactor of the methanol synthesis unit by the first syngas feed compressor.

In another embodiment, the particular feeding point of the reformer-based syngas to the inlet of the methanol synthesis unit, may be a position where it is combined with the overhead recycle stream of the methanol synthesis unit.

In an embodiment, said system (reforming system) is further arranged for said first stream being rich in propane and/or butane, and/or said second stream being an off-gas stream, being less than 15 wt % of said raw product from the gasoline synthesis section, or less than 15 wt % of said gasoline product stream.

The formation of such first and second streams in the gasoline synthesis plant represent less than 15 wt %, such as 10 wt % or less, for instance 5 wt % of the gasoline being produced, this being the raw product containing hydrocarbons boiling in the gasoline range, or the gasoline product stream. Despite the first and/or second streams only representing less than 15 wt % or less, e.g. about 10 wt %, or 5 wt %, of the hydrocarbon product, the first and/or second streams are advantageously reused in the plant or process to increase its overall efficiency: carbon (C-efficiency) and hydrogen efficiency (H-efficiency), instead of directing these stream away for use as fuel gas. Despite the low percentage of e.g. the first stream being rich in propane and/or butane, a dedicated reforming unit for reforming such by-product and off-gas into a syngas, is still advantageously provided, instead of utilizing it as said fuel gas.

More generally, the associated improvement in C-efficiency or overall plant/process efficiency is not merely equivalent to the percentage of said first and/or second streams with respect of hydrocarbon product, but higher; this being regardless of the percentage of e.g. LPG and/or off-gases being recycled with respect to hydrocarbon product, such as 10, 20, 30% or 40% of the gasoline being produced. For instance, where the formation of the first and/or second streams fed to the reforming system in the gasoline synthesis plant represents 10 wt %, the increase in overall plant/process efficiency is not merely 10%, but higher than 10%: in the e-SMR of the reforming system all carbon being fed is utilized for producing syngas, thereby providing CO into the inlet of the methanol synthesis unit, instead of the reforming system requiring the use of at least part of the gas, e.g. LPG, for burning purposes, thus as fuel gas, as it is conventional when operating with other type of reformers, such as autothermal reformers. Hence, not only is there an associated benefit of the provision of e-SMR in terms of drastically reducing or eliminating the carbon intensity of the plant by the e-SMR not emitting CO₂, but also C-efficiency and H-efficiency are increased, and not least methanol synthesis performance, e.g. MeOH loop performance is improved thus enabling a smaller methanol synthesis unit, as CO in the syngas from the reforming system is fed to the methanol synthesis.

In an embodiment, the methanol synthesis unit is arranged to provide an excess hydrogen stream, and said reforming system is arranged to receive at least a portion of said excess hydrogen stream from the methanol synthesis unit; optionally wherein said system (reforming system) comprises a hydrogenation section, and said hydrogenation section is arranged to receive said excess hydrogen stream.

In an embodiment, the methanol synthesis unit is a methanol synthesis loop. Accordingly, the methanol synthesis unit comprises:
optionally, a cleaning section, such as desulfurisation section, arranged to receive the CO₂ rich feed and the H₂ rich feed, or said syngas feed, thereby providing a cleaned methanol syngas feed, such as a desulfurized methanol syngas feed;
a methanol reactor arranged to receive said CO₂ rich feed and the H₂ rich feed, or said syngas feed, or the cleaned methanol syngas feed, such as the desulfurized methanol syngas feed, and to produce a raw methanol effluent stream;
a first separator arranged to receive the raw methanol effluent stream, and to produce i.e. to provide a bottom stream as said effluent stream comprising methanol, suitably after being fed to a second separator, such as low-pressure separator, from which an off-gas is generated; said first separator also being arranged to produce an overhead recycle stream to the methanol reactor;
a recycle compressor arranged to recycle the overhead recycle stream to the methanol reactor.

In an embodiment, the methanol synthesis unit further comprises:
means such as a mixing unit or junction to combine the overhead recycle stream with said CO₂ rich feed and/or said H₂ rich feed, or said syngas feed, i.e. the overhead recycle is provided in admixture with any of the above streams.

In an embodiment, the methanol synthesis unit may further comprise:
means such as a mixing unit or junction, suitably located downstream said recycle compressor, to combine any of the syngas streams from the system (reforming system), e.g. first syngas stream, with the overhead recycle stream.

The term “junction” may be used interchangeably with the term “juncture”. It denotes a mixing point.

Upstream the methanol reactor, as recited above, suitably as part of the methanol synthesis unit, there may be provided a cleaning section such as a desulfurisation section, for instance a sulfur absorber and sulfur guard, to remove sulfur from a syngas feed, since sulfur is detrimental for the downstream methanol reactor catalyst. By combining the syngas from the reforming system with the overhead recycle instead of directly with e.g. the syngas feed, the volumetric flow to the desulfurisation section remains unchanged, thus avoiding the penalty of increasing the sulfur removal capacity by providing a correspondingly larger desulfurisation section. After being combined with the overhead recycle, the syngas from the reforming system is then provided in admixture with said CO₂ rich feed and/or said H₂ rich feed, or in admixture with said syngas feed.

From the overhead recycle stream of the methanol synthesis unit, suitably upstream the recycle compressor, an optional fuel gas stream may be withdrawn from which a hydrogen stream is recovered. This hydrogen stream is herein referred to as said as excess hydrogen stream from the methanol synthesis unit, or simply “excess hydrogen stream”; and is for instance a hydrogen stream of a hydrogen recovery unit such as a pressure swing adsorption unit (PSA unit), i.e. a hydrogen recovery unit arranged to receive at least a portion of the overhead recycle stream as said fuel gas stream and provide said excess hydrogen stream; or a hydrogen stream of a purge gas scrubber, optionally together with a membrane unit, suitably arranged upstream the hydrogen recovery unit, e.g. PSA-unit. Thereby, additional hydrogen is internally produced in the plant or process. The hydrogen stream of the hydrogen recovery unit, e.g. a pressure swing adsorption (PSA) unit, is suitably sent to the reforming system, for instance to the hydrogenation section therein, or to the upgrading section of the plant, such as to the HDI reactor therein. The hydrogen stream of the purge gas scrubber and optional membrane unit may also be sent to e.g. the hydrogenation section of the reforming system of the plant. The hydrogenation section serves i.a. to remove any olefins being fed to the reforming system, as already recited.

Accordingly, in an embodiment, the methanol synthesis unit comprises:
a conduit for diverting a fuel gas stream as a portion of said overhead recycle stream to the methanol reactor;
a hydrogen recovery unit, such as any of a pressure swing adsorption (PSA) unit, gas scrubber, membrane unit, and combinations thereof, being arranged to receive at least a portion of said fuel gas stream and provide said excess hydrogen stream.

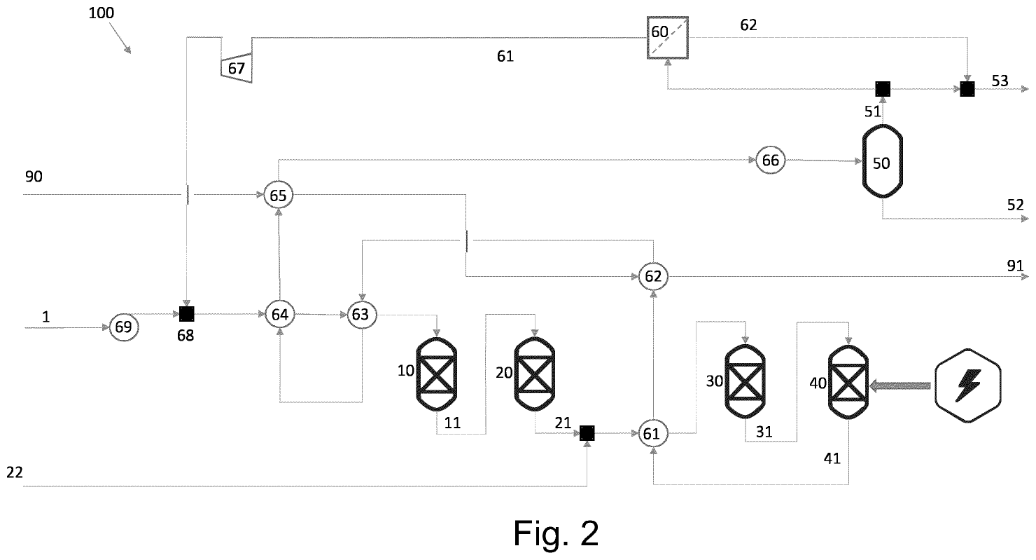

The provision of the excess hydrogen stream from the methanol synthesis unit enables also a simpler layout in the reforming system by eliminating the need of e.g. a hydrogen recovery section in the reforming system of the plant (such as membrane unit 60 in appended FIG. 2) to provide a hydrogen rich stream from the synthesis gas being produced and thereby also a hydrogen compressor to send the hydrogen rich stream to the hydrogenation section of the reforming system.

It would be understood that the plant further comprises a gasoline synthesis section, being arranged to receive at least a portion of the effluent stream comprising methanol from the methanol synthesis unit and provide a raw product containing hydrocarbons boiling in the gasoline range. Typically, the gasoline synthesis section is a MeOH to gasoline unit; the setup and operation of which is known in the art, cf. WO2008/071291 and WO 2016/116612.

The raw product from gasoline synthesis is upgraded to provide one or more commercial products. Therefore, the plant may further comprise said upgrading section, arranged to receive at least a portion of the raw product from the gasoline synthesis section, and provide a gasoline product stream. A first stream being rich in propane and/or butane, and/or a second stream, being off-gas stream are also produced in the upgrading section.

The second stream is an off-gas stream comprising CO₂, H₂, CH₄, and possibly higher hydrocarbons etc. The off-gas stream may comprise higher hydrocarbons, including ethane, propane, butane, pentane, olefins, oxygenates etc. In one aspect, off-gas stream comprises 20-40% CH4, 1-5% CO, 20-40% CO2, 5-15% H2, 10-20% higher hydrocarbons.

In an embodiment, said upgrading section comprises any of a HDI and HCR reactor, and is arranged to receive: a portion of the H₂ rich feed, and/or a portion of said excess hydrogen stream from the methanol synthesis unit.

Further integration is thereby achieved, as the hydrogen required is also sourced internally.

As noted, the gasoline synthesis plant further comprises the system (reforming system) as described herein. The system is arranged to receive at least a portion of said first stream from the upgrading section, and/or at least a portion of said second stream from the upgrading section, and provide a first syngas stream.

All details set out above relating to the system of the invention are equally applicable when the system is incorporated into the gasoline synthesis plant of the invention.

In the gasoline synthesis plant, certain syngas streams outputted from the system may be recycled upstream in the plant. Therefore, the plant further comprises a separation section, arranged to receive at least a portion of said first syngas stream and separate it into at least a second syngas stream and a process condensate, at least a portion of said second syngas stream is arranged to be fed to the inlet of the methanol synthesis unit, preferably in admixture with said CO₂ rich and/or said H₂ rich feed in one embodiment. In another embodiment, where the feed to the plant is derived from biomass gasification, said second syngas stream is arranged to be fed to the inlet of the methanol synthesis unit, preferably in admixture with said syngas feed from biomass gasification and optionally, H₂ rich feed.

Off-gas streams are generated in all MTG (methanol-to-gasoline) processes. One off-gas stream may come from the methanol loop. Other off-gas streams may come from the upgrading section downstream gasoline synthesis. Accordingly, in an embodiment, one or more of these additional off-streams in the plant may be arranged to be fed to the system, optionally in combination with first and/or said second stream. Suitably, as recited, a water separation unit is located between the methanol synthesis unit and the gasoline synthesis section, for instance upstream said methanol storage tank, and being arranged to remove water from the effluent stream comprising methanol.

In an embodiment, the gasoline synthesis plant does not comprise a reforming unit arranged upstream the methanol synthesis unit. Hence, there is no reforming unit for producing the syngas feed.

A process is also provided for reforming a first stream being rich in propane and/or butane (e.g. an LPG feed). This process comprises the steps of:
providing a system as described herein;
optionally, hydrogenating the first stream in the hydrogenation section to provide a hydrogenated first stream;
optionally, desulfurising said hydrogenated first stream in desulfurisation section, to provide a desulfurised first stream;
optionally, pre-reforming the first stream in pre-reforming section, to provide a pre-reformed first stream;
performing an electrical steam methane reforming (e-SMR) step on said first stream in an electrical steam methane reformer (e-SMR), to provide a first syngas stream.

An additional step in these processes may be the step of separating the first syngas stream in a separation section into at least a second syngas stream and a process condensate.

A process for gasoline synthesis from a CO₂ rich feed comprising CO₂, and a H₂ rich feed comprising H₂, is also provided, said process comprising the steps of:
providing a gasoline synthesis plant, as defined herein;
supplying CO₂ rich feed and H₂ rich feed to the methanol synthesis unit, and providing an effluent stream comprising methanol;
supplying at least a portion of the effluent stream comprising methanol from the methanol synthesis unit to the gasoline synthesis section, and providing a raw product containing hydrocarbons boiling in the gasoline range;
supplying at least a portion of the raw product from the gasoline synthesis section to the upgrading section, and providing a gasoline product stream; and a first stream being rich in propane and/or butane and/or a second feed being an off-gas stream;
supplying at least a portion of said first stream and/or said second stream from the upgrading section to said system, and providing a first syngas stream.

Further steps in this process include:
supplying at least a portion of the first syngas stream to the separation section, and separating it therein into at least a second syngas stream and a process condensate;
feeding at least a portion of said second syngas stream to the inlet of the methanol synthesis unit, preferably in admixture with said CO₂ rich feed and/or said H₂ rich feed.

A process for gasoline synthesis from a syngas feed from biomass gasification and optionally, a H₂ rich feed comprising H₂, is also provided, said process comprising the steps of:
providing a gasoline synthesis plant, as defined herein;
supplying syngas feed from biomass gasification and optionally, H₂ rich feed to the methanol synthesis unit, and providing an effluent stream comprising methanol;
supplying at least a portion of the effluent stream comprising methanol from the methanol synthesis unit to the gasoline synthesis section, and providing a raw product containing hydrocarbons boiling in the gasoline range;
supplying at least a portion of the raw product from the gasoline synthesis section to the upgrading section, and providing a gasoline product stream; and a first stream being rich in propane and/or butane, and/or a second feed being off-gas stream;
supplying at least a portion of said first stream and/or said second stream from the upgrading section as first stream and/or second stream respectively to said system, and providing a first syngas stream.

Further steps in this process include:
supplying at least a portion of the first syngas stream to separation section, and separating it therein into at least a second syngas stream and a process condensate;
feeding at least a portion of said second syngas stream to the inlet of the methanol synthesis unit, preferably in admixture with said syngas feed and/or said H₂ rich feed.

Overall, in the illustrated process/system, first feed (e.g. a LPG feed) and/or second feed (i.e. off-gas stream) are hydrogenated, desulfurized and prereformed before sending it to e-SMR. The effluent stream gets cooled in series of heat exchangers by pre-reformer feed preheat, steam generation in waste heat boiler, feed preheater, LPG feed vaporizer, preheating of boiler feed water etc. The water in the effluent stream gets condensed and then separated. A part of syngas is then used for H₂ recovery for internal use for hydrogenation and pre-reforming. The rest of the syngas is sent to the MeOH loop.

### Specific Embodiments

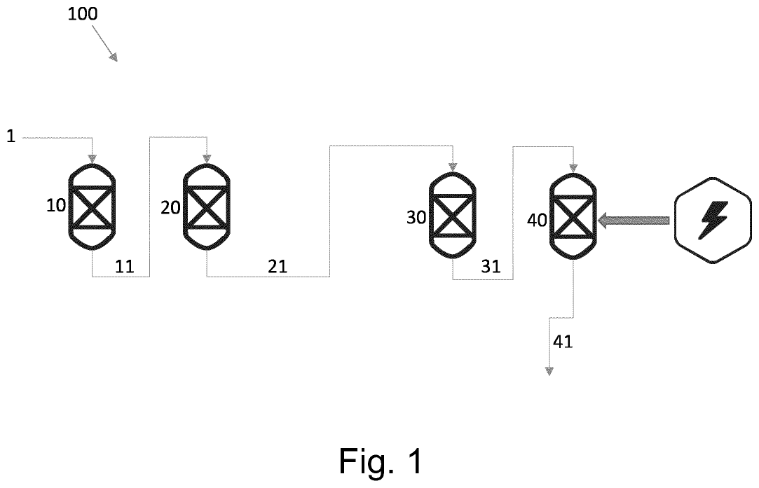

FIG. 1 shows a simple layout of one embodiment of the system 100. Throughout the embodiments in the figures, the first propane and/or butane rich stream is an LPG stream. LPG stream 1 is hydrogenated in hydrogenation section 10 to provide a hydrogenated LPG stream 11. This hydrogenated LPG stream 11 is desulfurised in desulfurisation section 20, to provide a desulfurised LPG stream 21. The desulfurised LPG stream 21 is pre-reformed in pre-reforming section 30, to provide a pre-reformed stream 31. Electrical steam methane reforming (e-SMR) is performed on the pre-reformed stream 31 in electrical steam methane reformer (e-SMR, 40), for which electrical power is illustrated by the “lightning” symbol, to provide a first syngas stream (41).

FIG. 2 shows a more developed layout of the system 100. LPG feed 1 is compressed in first pump 69. The compressed LPG feed is-in this layout-mixed with hydrogen rich stream 61 at mixer 68 before being passed through heat exchangers 64, 63 to heat exchange with the first syngas stream 41. The heated LPG feed is hydrogenated in hydrogenation section 10 to provide a hydrogenated LPG stream 11 which is subsequently desulfurised in desulfurisation section 20, to provide a desulfurised LPG stream 21. Desulfurised LPG stream 21 may be mixed with process steam 22, and the mixed stream is again heat exchanged with the first syngas stream 41.

The desulfurised LPG stream 21 is pre-reformed in pre-reforming section 30, to provide a pre-reformed stream 31. Electrical steam methane reforming (e-SMR) is performed on the pre-reformed stream 31 in electrical steam methane reformer (e-SMR, 40), to provide a first syngas stream 41.

First syngas stream 41 is then heat exchanged with boiler feed water 90 in waste heat boiler 62, providing export steam 91. Subsequently, first syngas stream 41 is passed through heat exchangers 64, 63 (as noted above), and then heat-exchanged once more with boiler feed water 90 in heat exchanger 65. Additional cooling takes place in cooling unit 66.

The first syngas stream 41 is passed to a separation section 50 where it is separated into at least a second syngas stream 51 and a process condensate 52. A portion of the second syngas stream 51 is passed to hydrogen recovery section 60, where a hydrogen-rich stream 61 is separated and a third syngas stream 62 is provided. The hydrogen-rich stream 61 is compressed at compressor 67, and then combined with the LPG feed 1, upstream the hydrogenation section 10 (as noted above).

A portion of the second syngas stream 51 and a portion of the third syngas stream 62 are combined to a combined syngas stream 53.

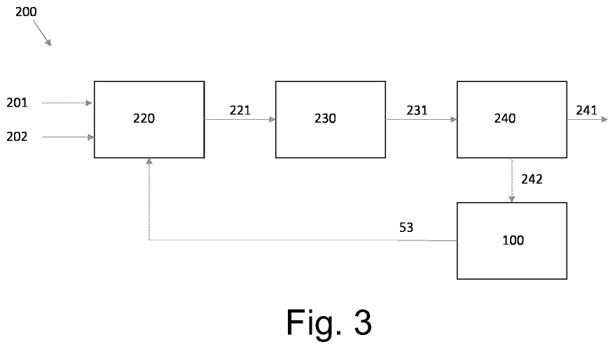

FIG. 3 shows a gasoline synthesis plant 200 according to the invention. A system 100, as per FIGS. 1-2 is provided to make recycling of LPG possible. In the plant 200 of FIG. 3, a CO₂ rich feed 201 comprising CO₂ and a H₂ rich feed 202 comprising H₂ are sent to methanol synthesis unit 220, from which an effluent stream 221 comprising methanol is provided. This effluent stream 221 is supplied to gasoline synthesis section 230, and a raw product 231 containing hydrocarbons boiling in the gasoline range is provided. This raw product 231 is fed to an upgrading section 240, where it is upgraded to a gasoline product stream 241 and an LPG stream 242. The resulting LPG stream 242 is fed to a system 100 as described above, and a second syngas stream 53 is provided, which is then recycled to the methanol synthesis unit 220.

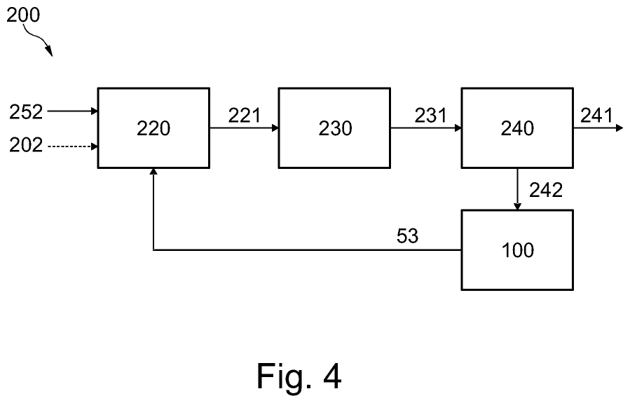

FIG. 4 shows a gasoline synthesis plant 200 according to the invention. A system 100, as per FIGS. 1-2 is provided to make recycling of LPG possible. In the plant 200 of FIG. 3, a biogas feed 252 and an optional H₂ rich feed 202 comprising H₂ are sent to methanol synthesis unit 220, from which an effluent stream 221 comprising methanol is provided. This effluent stream 221 is supplied to gasoline synthesis section 230, and a raw product 231 containing hydrocarbons boiling in the gasoline range is provided. This raw product 231 is fed to an upgrading section 240, where it is upgraded to a gasoline product stream 241 and an LPG stream 242. The resulting LPG stream 242 is fed to a system 100 as described above, and a second syngas stream 53 is provided, which is then recycled to the methanol synthesis unit 220.

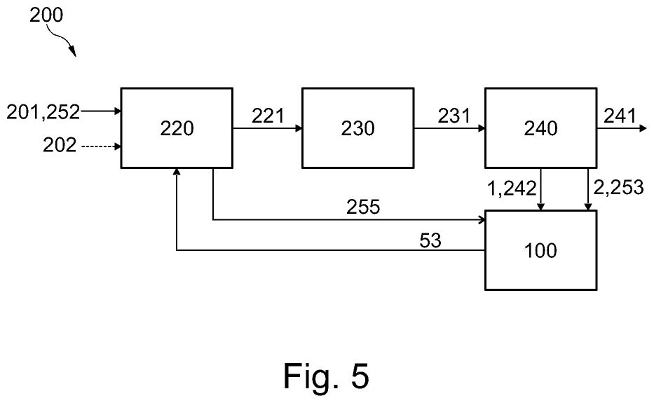

FIG. 5 shows a gasoline plant 200 according to the invention. From the upgrading section 240, the first stream 1, 242 being rich in propane and/or butane, e.g. LPG, and a second stream 2, 253 being an off-gas stream, are fed to the to the system 100 (reforming system). Although shown as independent streams in FIG. 5, these streams may be combined into a single inlet to the reforming system; hence, the second stream 2, 253 being an off-gas stream comprising CO₂, H₂ and CH₄, is suitably arranged to be mixed with the first stream 1, 242 upstream the inlet of the e-SMR 40. The methanol synthesis unit 220 is further arranged to provide an excess hydrogen stream 255, and the reforming system 100 is arranged to receive at least a portion of this excess hydrogen stream 255, for instance by providing it to the hydrogenation section 10 therein.

### Example 1

TABLE 1
Comparison of product yield by LPG recycle
in biomass-to-gasoline plant

| Paremeters | UoM | C1 | C2 |
|---|---|---|---|
| Main feed | — | Syngas from biomass gasification | |
| eSMR | — | No | Yes |
| Syngas feed mass flow | % | 100 | 100 |
| LPG byproduct mass flow | % | 100 | 121 |
| Methanol mass flow | % | 100 | 122 |
| Gasoline mass flow | % | 100 | 122 |

Results from biomass-to-gasoline plant is shown in Table 1. The main feed is the syngas from biomass gasification. No other feed is used. C1 is the case, where LPG and off-gas byproducts from the system are not utilized. In C2, all LPG and off-gas streams are recycled and reformed in e-SMR to produce additional syngas and then added to main syngas feed to the methanol synthesis loop. As a result, intermediate methanol production is increased by 22%. The final gasoline product is also increased by 22% highlight significantly better yield of product from same amount of feed. As compared to the use of a fired reformer, the use of an e-SMR eliminates the need for any removal of CO₂ formed by fuel firing. Further, the carbon in the LPG is advantageously converted to CO in the syngas produced. Overall emission from such process is negligible through purge from methanol loop. The extent of this emission depends solely on the impurities in the main feed.

The present invention has been described with reference to a number of embodiments and figures. However, the skilled person is able to select and combine various embodiments within the scope of the invention, which is defined by the appended claims. All documents referenced herein are incorporated by reference.

We claim:

1. A system for reforming a first propane and/or butane rich stream, said system comprising:
a first stream, being rich in propane and/or butane;
an electrical steam methane reformer (e-SMR,), arranged to receive the first stream and carry out an electrical steam methane reforming (e-SMR) step, so as to provide a first syngas stream.

2. The system according to claim 1, wherein the first stream is an LPG stream.

3. The system according to claim 1, further comprising a second stream being an off-gas stream comprising CO₂, H₂ and CH₄, said second stream being arranged to be mixed with the first stream, upstream of an inlet of the e-SMR.

4. The system according to claim 1, further comprising a separation section, arranged to receive at least a portion of said first syngas stream and separate it into at least a second syngas stream and a process condensate.

5. The system according to claim 1, further comprising:
a hydrogenation section for hydrogenating the first stream to provide a hydrogenated first stream;
wherein the electrical steam methane reformer (e-SMR,) is arranged to receive: the first stream, or the hydrogenated first stream; and carry out said electrical steam methane reforming (e-SMR) step, so as to provide said first syngas stream.

6. The system according to claim 5, further comprising one or more heat exchangers, being arranged to provide heat exchange between the first syngas stream and one or more of: the first stream, a desulfurized first stream and a boiler feed water stream.

7. The system according to claim 4, further comprising a hydrogen recovery section, said hydrogen recovery section being arranged to receive at least a portion of the second syngas stream and provide at least a hydrogen-rich stream and a third syngas stream; wherein at least a portion of the hydrogen-rich stream is arranged to be combined with the first stream and/or off-gas stream, upstream the hydrogenation section; and/or wherein at least a portion of the second syngas stream and at least a portion of the third syngas stream are arranged to be combined to form a combined syngas stream.

8. A system for reforming a first propane and/or butane rich stream, said system comprising:
a first stream, being rich in propane and/or butane, wherein said first stream comprises at least 50% propane and/or butane;
an electrical steam methane reformer (e-SMR,), arranged to receive the first stream and carry out an electrical steam methane reforming (e-SMR) step, so as to provide only a single product stream, wherein the product stream is a first syngas stream.

9. A process for reforming a first stream being rich in propane and/or butane, said process comprising the steps of:
providing a system according to claim 1; and
performing an electrical steam methane reforming (e-SMR) step on said first stream in an electrical steam methane reformer (e-SMR,), to provide the first syngas stream.

10. A gasoline synthesis plant, comprising:
a CO₂ rich feed supplying CO₂ to said plant,
a H₂ rich feed supplying H₂ to said plant,
a methanol synthesis unit, arranged to receive the CO₂ rich feed and the H₂ rich feed, and provide an effluent stream comprising methanol;
a gasoline synthesis section, arranged to receive at least a portion of the effluent stream, and provide a raw product containing hydrocarbons boiling in the gasoline range;
an upgrading section, arranged to receive at least a portion of the raw product from the gasoline synthesis section, and provide a gasoline product stream; and a first stream being rich in propane and/or butane, and/or a second stream being an off-gas stream;
said gasoline synthesis plant further comprising the system according to claim 1,
wherein said system is arranged to receive at least a portion of said first stream from the upgrading section,
and/or wherein said system is arranged to receive at least a portion of said second stream from the upgrading section,
and provide a first syngas stream from said first and/or said off-stream,
wherein the plant further comprises in said system a separation section, arranged to receive at least a portion of said first syngas stream and separate it into at least a second syngas stream and a process condensate, and wherein at least a portion of said second syngas stream is arranged to be fed to an inlet of the methanol synthesis unit.

11. A gasoline synthesis plant, comprising:
a syngas feed from biomass gasification supplying said plant,
a methanol synthesis unit, arranged to receive the syngas feed;
a gasoline synthesis section, arranged to receive at least a portion of the effluent stream comprising methanol, and provide a raw product containing hydrocarbons boiling in the gasoline range;
an upgrading section, arranged to receive at least a portion of the raw product from the gasoline synthesis section, and provide a gasoline product stream; and a first stream being rich in propane and/or butane, and/or a second stream being an off-gas stream;
said gasoline synthesis plant further comprising the system according to claim 1,
wherein said system is arranged to receive at least a portion of said first stream from the upgrading section,
and/or wherein said system is arranged to receive at least a portion of said second stream from the upgrading section,
and provide the first syngas stream from said first and/or said second stream,
wherein the plant further comprises a separation section, arranged to receive at least a portion of said first syngas stream and separate it into at least a second syngas stream and a process condensate, and wherein at least a portion of said second syngas stream is arranged to be fed to the inlet of the methanol synthesis unit.

12. The gasoline synthesis plant according to claim 10, wherein a methanol storage tank is arranged between said methanol synthesis unit and said gasoline synthesis section for storing at least a portion of the effluent stream.

13. The gasoline synthesis plant according claim 10, wherein said system is further arranged for said first stream being rich in propane and/or butane, and/or said second stream being an off-gas stream, being less than 15 wt % of said raw product from the gasoline synthesis section, or less than 15 wt % of said gasoline product stream.

14. The gasoline synthesis plant according to claim 10, wherein the methanol synthesis unit is arranged to provide an excess hydrogen stream, and wherein said reforming system is arranged to receive at least a portion of said excess hydrogen stream.

15. The gasoline synthesis plant according to claim 10, wherein the methanol synthesis unit is a methanol synthesis loop, which comprises:
a methanol reactor arranged to receive said CO₂ rich feed and the H₂ rich feed, or said syngas feed, to produce a raw methanol effluent stream;
a first separator arranged to receive the raw methanol effluent stream, and to produce a bottom stream as said effluent stream comprising methanol; said first separator also being arranged to produce an overhead recycle stream to the methanol reactor;
a recycle compressor arranged to recycle the overhead recycle stream to the methanol reactor.

16. The gasoline synthesis plant according to claim 15, wherein the methanol synthesis unit comprises:
a conduit for diverting a fuel gas stream as a portion of said overhead recycle stream;
a hydrogen recovery unit, being arranged to receive at least a portion of said fuel gas stream and provide said excess hydrogen stream.

17. The gasoline synthesis plant, according to claim 10, wherein one or more additional off-streams in the plant are arranged to be fed to the system.

18. The gasoline synthesis plant according to claim 10, wherein a water separation unit is located between the methanol synthesis unit and the gasoline synthesis section, and being arranged to remove water from the effluent stream comprising methanol.

19. The gasoline synthesis plant according to claim 10, which does not comprise a reforming unit arranged upstream of the methanol synthesis unit.

20. A process for gasoline synthesis from a CO₂ rich feed comprising CO₂, and a H₂ rich feed comprising H₂, said process comprising the steps of:
providing a gasoline synthesis plant, according to claim 10;
supplying CO₂ rich feed and H₂ rich feed to the methanol synthesis unit, and providing an effluent stream comprising methanol;
supplying at least a portion of the effluent stream comprising methanol from the methanol synthesis unit to the gasoline synthesis section, and providing a raw product containing hydrocarbons boiling in the gasoline range;
supplying at least a portion of the raw product from the gasoline synthesis section to the upgrading section, and providing a gasoline product stream; and a first stream being rich in propane and/or butane, and/or a second stream being an off-gas stream;
supplying at least a portion of said first stream and/or said second stream from the upgrading section to said system, and providing a first syngas stream;
supplying at least a portion of said first syngas stream to separation section, and separating it therein into at least a second syngas stream and a process condensate;
feeding at least a portion of said second syngas stream to the inlet of the methanol synthesis unit.

21. A process for gasoline synthesis from a syngas feed from biomass gasification, and, optionally, a H₂ rich feed comprising H₂, said process comprising the steps of:
providing a gasoline synthesis plant, according to claim 11;
supplying syngas feed from biomass gasification to the methanol synthesis unit, and providing an effluent stream comprising methanol;
supplying at least a portion of the effluent stream comprising methanol from the methanol synthesis unit to the gasoline synthesis section, and providing a raw product containing hydrocarbons boiling in the gasoline range;
supplying at least a portion of the raw product from the gasoline synthesis section to the upgrading section, and providing a gasoline product stream; and a first stream being rich in propane and/or butane, and/or a second stream being an off-gas stream;
supplying at least a portion of said first stream and/or said second stream from the upgrading section to said system, and providing a first syngas stream;
supplying at least a portion of said first syngas stream to separation section (50), and separating it therein into at least a second syngas stream and a process condensate;
feeding at least a portion of said second syngas stream to the inlet of the methanol synthesis unit.

22. The system according to claim 5, further comprising:
a desulfurization section for desulfurizing said hydrogenated first stream to provide a desulfurized first stream; and
a pre-reforming section for pre-reforming the first stream to provide a pre-reformed first stream;
wherein the electrical steam methane reformer (e-SMR,) is further arranged to receive: the desulfurized first stream, or the pre-reformed first stream; and carry out said electrical steam methane reforming (e-SMR) step, so as to provide said first syngas stream.

23. The process according to claim 9, further comprising, prior to the electrical steam methane reforming:
hydrogenating the first stream in a hydrogenation section;
desulfurizing the first stream in a desulfurization section; and/or
pre-reforming the first stream in a pre-reforming section.

24. The gasoline synthesis plant of claim 10, wherein said upgrading section comprises:
a de-ethanizer for providing at least a portion of said second stream, an LPG splitter for providing said first stream, a hydroisomerisation (HDI) reactor, and/or a hydrocracking (HCR) reactor; and
wherein the at least a portion of said second syngas stream is fed to the inlet of the methanol synthesis unit in admixture with said CO₂ rich feed and/or said H₂ rich feed.

25. The gasoline synthesis plant of claim 11, further comprising:
an H₂ rich feed comprising H₂ supplying said plant;
wherein the methanol synthesis unit is arranged to receive the H₂ rich feed in addition to the syngas feed, and provide the effluent stream comprising methanol;
wherein said upgrading section comprises: a de-ethanizer for providing at least a portion of said second stream, an LPG splitter for providing said first stream, a hydroisomerisation (HDI) reactor, and a hydrocracking (HCR) reactor; and
wherein the at least a portion of said second syngas stream is arranged to be fed to the inlet of the methanol synthesis unit in admixture with said syngas feed and/or said H₂ rich feed.

26. The gasoline synthesis plant according to claim 14, wherein said system comprises a hydrogenation section, and said hydrogenation section is arranged to receive said excess hydrogen stream.

27. The gasoline synthesis plant according to claim 15, wherein the methanol synthesis loop comprises:
a cleaning section comprising a desulfurization section, arranged to receive the CO₂ rich feed and the H₂ rich feed, or said syngas feed, thereby providing a cleaned methanol syngas feed, wherein the cleaned methanol syngas feed is a desulfurized methanol syngas feed;

28. The gasoline synthesis plant according to claim 16, wherein the hydrogen recovery unit comprises at least one of: a pressure swing adsorption (PSA) unit, a gas scrubber, a membrane unit, and combinations thereof.

29. The process for gasoline synthesis according to claim 20, wherein the step of feeding at least a portion of said second syngas stream to the inlet of the methanol synthesis unit comprises feeding the at least a portion of said second syngas stream to the inlet of the methanol synthesis unit in admixture with said syngas feed and/or said H₂ rich feed.

30. The process for gasoline synthesis according to claim 21, comprising supplying an H₂ rich feed to the methanol synthesis unit, in addition to the syngas feed from biomass gasification; and
wherein the step of feeding at least a portion of said second syngas stream to the inlet of the methanol synthesis unit comprises feeding the at least a portion of said second syngas stream to the inlet of the methanol synthesis unit in admixture with said syngas feed and/or said H₂ rich feed.

31. A process for reforming a first stream being rich in propane and/or butane, said process comprising the steps of:
providing a system according to claim 8; and
performing an electrical steam methane reforming (e-SMR) step on said first stream in an electrical steam methane reformer (e-SMR,), to provide the first syngas stream.

32. A gasoline synthesis plant, comprising:
a CO₂ rich feed supplying CO₂ to said plant;
a H₂ rich feed supplying H₂ to said plant;
a methanol synthesis unit, arranged to receive the CO₂ rich feed and the H₂ rich feed, and provide an effluent stream comprising methanol;
a gasoline synthesis section, arranged to receive at least a portion of the effluent stream, and provide a raw product containing hydrocarbons boiling in the gasoline range;
an upgrading section, arranged to receive at least a portion of the raw product from the gasoline synthesis section, and provide a gasoline product stream; and a first stream being rich in propane and/or butane, and/or a second stream being an off-gas stream;
said gasoline synthesis plant further comprising the system according to claim 8, wherein said system is arranged to receive at least a portion of said first stream from the upgrading section;
and/or wherein said system is arranged to receive at least a portion of said second stream from the upgrading section and provide a first syngas stream from said first and/or said off-gas stream; and
wherein the plant further comprises in said system a separation section arranged to receive at least a portion of said first syngas stream and separate it into at least a second syngas stream and a process condensate, and wherein at least a portion of said second syngas stream is arranged to be fed to an inlet of the methanol synthesis unit.

33. A gasoline synthesis plant, comprising:
a syngas feed from biomass gasification supplying said plant;
a methanol synthesis unit, arranged to receive the syngas feed;
a gasoline synthesis section, arranged to receive at least a portion of the effluent stream comprising methanol, and provide a raw product containing hydrocarbons boiling in the gasoline range;
an upgrading section, arranged to receive at least a portion of the raw product from the gasoline synthesis section, and provide a gasoline product stream; and a first stream being rich in propane and/or butane, and/or a second stream being an off-gas stream;
said gasoline synthesis plant further comprising the system according to claim 8, wherein said system is arranged to receive at least a portion of said first stream from the upgrading section;
and/or wherein said system is arranged to receive at least a portion of said second stream from the upgrading section and provide the first syngas stream from said first and/or said second stream; and
wherein the plant further comprises a separation section, arranged to receive at least a portion of said first syngas stream and separate it into at least a second syngas stream and a process condensate, and wherein at least a portion of said second syngas stream is arranged to be fed to the inlet of the methanol synthesis unit.
